(19) United States
(12) Patent Application Publication
(10) Pub. No.: US 2020/0188177 A1
(43) Pub. Date: Jun. 18, 2020

(54) DERMAL DEVICE FOR ADMINISTRATION OF ONE OR MORE ACTIVE AGENTS TO THE SKIN

(71) Applicant: Mylan Inc., Canonsburg, PA (US)

(72) Inventors: Gaurav Thakersi TOLIA, Morgantown, WV (US); Malachi PLACHTA, Morgantown, WV (US); Grant HENDERSON, St. Albans, VT (US); Brad BARNETT, St. Albans, VT (US)

(73) Assignee: Mylan Inc., Canonsburg, PA (US)

(21) Appl. No.: 16/803,532

(22) Filed: Feb. 27, 2020

Related U.S. Application Data
(63) Continuation of application No. 15/096,953, filed on Apr. 12, 2016, now abandoned.

(51) Int. Cl. A61F 13/00; A61F 13/40; A61F 13/02

(52) U.S. Cl. CPC A61F 13/00; A61F 13/0246; A61F 13/022; A61M 35/006

(57) ABSTRACT

A double disk dermal device for administration of one or more active agents to the skin or mucosa of a host, in particular a patch is described including: a) active reservoir film layer; b) a backing overlay adjacent to the active reservoir film layer extending beyond the perimeter of the reservoir layer in all directions; c) second overlay backing layer with a coating of pressure sensitive adhesive which is adjacent to the first backing overlay extending beyond the perimeter of the first backing overlay in all directions; and d) a removable release liner. A method of making the device is also provided.

### CROSS REFERENCE TO RELATED APPLICATION

This application is a continuation of U.S. application Ser. No. 15/096,953 filed Apr. 12, 2016, the entire contents of which are incorporated herein by reference.

### BACKGROUND

### 1. Field of the Invention

The present invention relates in general to a device for release of active agents to be administered to the skin or mucosa of a host. More particularly, the present invention relates to transdermal patches that minimize migration of active agent. This invention further relates to methods of manufacturing the devices in a continuous manufacturing process.

### 2. Description of Related Art

Dermal devices are adhesive patches available as transdermal patches, topical patches or plasters intended to be applied on the skin for the administration of a wide variety of active agents into or through the skin. Transdermal patches are dermal devices where the active agents are required to be delivered through the skin into the systemic circulation. Topical patches are dermal devices applied on the skin for delivery of pharmacologically or cosmetic active agents into or through skin for local effect.

Such dermal devices typically require an adhesive system to hold the patch on the skin for the required period of administration and in some cases contain excipients for a variety of reasons for example either to enhance delivery of active agent, to increase adhesion, provide counter-irritant properties, or to act as a plasticizer or softening agent. The period of administration of dermal devices could vary depending on the active agent being delivered and could range from a few hours to several days of application. In some cases, dermal devices are required for several days of administration and a double disk configuration dermal device is preferred to ensure adhesion of the device on the skin without the patch lifting off from the edges or falling off from the site of administration. Generally, “double disk” dermal devices consist of an overlay outer adhesive patch termed an outer disk attached to a smaller inner active reservoir patch separated from the outer disk by a backing of the same size as the inner active reservoir patch. The inner and outer disks are assembled together on a packaging line to form the double disk finished systems. In such double disk systems, the inner disk reservoir film comes in contact with the outer disk adhesive layer allowing either active agent or excipients incorporated in the inner disk to migrate over time.

Such a double disk configuration patch, where the inner active reservoir is in contact with the outer adhesive overlay could lead to migration of active agent or excipients from the inner disk into the outer disk, thereby adversely affecting product characteristics such as efficacy, adhesion, drug release or delivery profile over its shelf-life. In order to minimize the changes that could occur in a double disk device configuration system, alternate double disk configurations where the inner active reservoir is separated from the outer adhesive overlay during the shelf-life of the product have been proposed. Alternate solutions to the double disk configuration have been described by either incorporating a releasable barrier layer or incorporating an impermeable backing that extends in all direction from the inner active reservoir film.

Double disk configurations where a releasable barrier layer is used, requires additional steps to be performed by the user of the device in order to apply the device on the skin. Increasing the number of steps for use of a medical product by patients could lead to either a decrease in patient compliance or an increase in user error.

Manufacturing of a double disk configuration containing an impermeable backing extending in all directions from the active reservoir film as a continuous process is difficult to achieve due to the adhesive nature of the dermal device and due to the lack of ability to kiss-cut, separate, and wind the adhesive waste from either a release liner when the laminate is self-wound or from substrates that do not have a release coating on it.

Such devices and methods are illustrative of those that can be achieved by the various embodiments disclosed herein and are not intended to be exhaustive or limiting of the possible advantages that can be realized. Thus, these and other embodiments will be apparent from the description herein or can be learned from practicing the various exemplary embodiments, both as embodied herein or as modified in view of any variation that may be apparent to those skilled in the art. Accordingly, the present invention resides in the novel methods, arrangements, combinations, and improvements herein shown and described in various exemplary embodiments.

### SUMMARY

In light of the present need for improved devices and methods for manufacturing such devices, a brief summary of various embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

The present invention relates to dermal devices that employ a physical barrier between the inner active reservoir layer and the outer adhesive layer for minimizing the migration of active agents or excipients. In addition, this invention provides methods of producing these dermal devices using a continuous manufacturing process. More specifically, the devices of this invention are double disk in nature and include an inner disk containing volatile or non-volatile active agent or agents and/or excipients, separated by a physical barrier provided by an impermeable backing membrane which extends on all sides from the active inner disk, an adhesive outer disk and a removable release liner.

Various embodiments relate to a double disk configuration dermal device where the inner active reservoir layer contains a porous mesh film that brings structural integrity to the coated and dried active reservoir layer allowing continuous kiss-cutting and stripping of inner disk film waste followed by lamination onto a backing film and additional kiss-cutting to provide a physical barrier between inner active and outer adhesive layer.

Various embodiments relate to a double disk configuration dermal device where the single or multi-layer inner active reservoir contains a non-porous rate controlling film which in addition to providing a rate controlling function, brings structural integrity to the coated and dried active reservoir layer allowing continuous kiss-cutting and stripping of inner active disk waste followed by lamination onto a backing film and additional kiss-cutting, to provide a physical barrier between inner active and outer adhesive layer.

Various embodiments relate to a double disk configuration dermal device where the single or multi-layer inner active reservoir is laminated to a porous mesh film that brings structural integrity to the coated and dried active reservoir layer allowing continuous kiss-cutting and stripping of inner disk active waste followed by lamination onto a backing film and additional kiss-cutting, to provide a physical barrier between inner active and outer adhesive layer.

Various embodiments relate to a double disk configuration dermal device where the inner disk active reservoir contains a backing layer of the same size as the active inner reservoir layer and an adhesive tie layer on the side away from skin, which allows for continuous manufacturing, and upon kiss-cutting, laminating with a second backing, followed by additional kiss-cutting to create a physical backing barrier between the inner and outer layers.

Various embodiments relate to a double disk configuration dermal device where the inner disk active reservoir has a peripheral ring of backing separating the inner disk from the outer disk layer.

Various embodiments relate to a continuous manufacturing process including the steps of: a) coating an active adhesive film on a release liner, b) drying or curing to fix the adhesive film on the release liner, c) laminating with a second release liner capable of providing differential release or self-winding the adhesive film to the first release liner with differential release coating; d) slitting the active adhesive film into suitable size strips; e) removing the second release liner on the packaging line; f) die cutting or kiss-cutting the adhesive film on the first release liner, g) picking the skeleton waste using a structurally rigid planar membrane; h) laminating with a backing film of suitable width; i) die-cutting or kiss-cutting the backing film of a size bigger than the adhesive film; j) overlaying the outer disk adhesive film over the kiss-cut inner active adhesive laminate; k) die-cutting or kiss cutting the outer disk adhesive film; l) performing a guillotine cut of the first release liner to obtain a finished double disk system; and m) pouching the finished double disk systems.

Various embodiments relate to a continuous manufacturing process including the steps of: a) coating an active adhesive film on a release liner, b) drying or curing to fix the adhesive film on the release liner, c) placing a porous mesh film on the adhesive film; d) winding the adhesive film with the porous mesh film on a second release liner or self-winding the adhesive film on the first release liner, e) slitting the adhesive film with mesh film into suitable size strips; f) removing the second release liner on the packaging line; g) die-cutting or kiss-cutting the adhesive film on the first release liner, h) laminating with a backing film of suitable width; i) die-cutting or kiss-cutting the backing film of the size bigger than the adhesive film; k) overlaying the outer disk adhesive film over the kiss-cut adhesive laminate; l) die cutting or kiss cutting the outer disk adhesive film; m) performing a guillotine cut of the first release liner to obtain finished double disk systems; and n) pouching the finished double disk systems.

Various embodiments relate to a continuous manufacturing process including the steps of: a) coating an adhesive film on a release liner, b) positioning a porous mesh film on the wet adhesive film; c) drying or curing to fix the adhesive film with porous mesh film on the release liner, d) winding the adhesive film with the porous mesh film on a second release liner, e) slitting the adhesive film with mesh film into suitable size strips; f) removing the second release liner on the packaging line; g) die-cutting or kiss-cutting of the adhesive film on the first release liner, h) laminating with a backing film of suitable width; i) die-cutting or kiss-cutting the backing film of the size bigger than the adhesive film; j) overlaying the outer disk adhesive film over the kiss-cut adhesive laminate; k) die-cutting or kiss-cutting the outer disk adhesive film; l) performing a guillotine cut of the first release liner to obtain finished double disk systems; and m) pouching the finished double disk systems.

Various embodiments relate to a continuous manufacturing process including the steps of: a) coating an adhesive film on a release liner with a porous mesh film laid on top of the release liner, b) drying or curing to fix the adhesive film with porous mesh film on the release liner, c) winding the adhesive film with the porous mesh film on a second release liner, d) slitting the adhesive film with mesh film into suitable size strips; e) removing the second release liner on the packaging line; f) die-cutting or kiss-cutting of the adhesive film on the first release liner, g) laminating with a backing film of suitable width; h) die-cutting or kiss-cutting the backing film of the size bigger than the adhesive film; i) overlaying the outer disk adhesive film over the kiss-cut adhesive laminate; j) die-cutting or kiss-cutting the outer disk adhesive film; k) performing a guillotine cut of the first release liner to obtain finished double disk systems; and l) pouching the finished double disk systems.

Various embodiments relate to a continuous manufacturing process including the steps of: a) coating an active adhesive film on a release liner, b) drying or curing to fix the active adhesive film on the release liner, c) laminating with a backing film laminate coated with an adhesive tie layer on the backing side, wherein the backing film laminate contains a backing film, adhesive tie layer and a release liner, d) slitting the adhesive film laminate into suitable size strips; e) rewinding the release liner attached to the tie layer adhesive; f) die-cutting or kiss-cutting the adhesive film on the first release liner, g) laminating with a second backing of suitable width on the exposed adhesive tie layer, h) die-cutting or kiss-cutting the second backing film of the size bigger than the adhesive film; i) overlaying the outer disk adhesive film over the kiss-cut adhesive laminate; j) die-cutting or kiss-cutting the outer disk adhesive film of the size bigger than the active inner disk; k) performing a guillotine cut of the first release liner to obtain finished double disk systems; and l) pouching the finished double disk systems.

### BRIEF DESCRIPTION OF THE DRAWINGS

In order to better understand various exemplary embodiments, reference is made to the accompanying drawings, wherein

FIG. 1 illustrates a planar view of a double disk dermal device.

FIG. 2 illustrates an exploded cross-sectional view of the device shown in FIG. 1.

FIG. 3 illustrates an exploded cross-section of a dermal device of double disk configuration.

FIG. 4 illustrates an unassembled exploded view of the components of the device shown in FIG. 3.

FIG. 5 illustrates an exploded cross-section of a dermal device of double disk configuration.

FIGS. 6a and 6b each illustrate an exploded cross-section of a dermal device of double disk configuration containing an adhesive tie layer with and without a porous membrane, respectively.

FIG. 7 illustrates an exploded cross-section of a dermal device of double disk configuration in accordance.

FIGS. 8-12 are flow diagrams that illustrate methods of manufacturing double disk devices.

FIG. 13 illustrates the process of drying of the active adhesive layer and laminating the active adhesive layer with a porous membrane.

FIG. 14 illustrates the process of drying the active adhesive layer formed as a wet blend with the porous membrane.

FIG. 15 illustrates the process of drying the active inner reservoir layer using a pre-cut template laminate.

FIG. 16 illustrates an exemplary production method.

### DETAILED DESCRIPTION

The present disclosure relates generally to dermal devices of double disk configuration comprising an outer disk overlay layer, which includes a backing and pressure sensitive adhesive coating, separated from the active inner disk reservoir film with a backing layer that extends in all directions of the active inner disk reservoir film and methods of making the same.

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., or), unless otherwise indicated (e.g., “or else” or “or in the alternative”). Additionally, the various embodiments described herein are not necessarily mutually exclusive and may be combined to produce additional embodiments that incorporate the principles described herein.

The present invention is based on the finding that adhesive coated films do not have sufficient tensile strength to allow for kiss-cutting and waste rewinding in a continuous manufacturing process in order to make a double disk device configuration. It has surprisingly been found that a porous mesh membrane or a non-porous rate-controlling membrane would provide the required strength for allowing continuous manufacturing of these devices. As used herein, the term “continuous” refers to a manufacturing process wherein the process does not involve a stoppage for removal or stripping of adhesive or backing film skeletons.

One aspect of the invention features a dermal device where the inner active reservoir layer is dried during coating with a porous mesh film or a non-porous rate controlling membrane of sufficient structural integrity that would allow for continuous kiss-cutting and waste rewinding before lamination with a backing film. This feature provided by the porous mesh film and the non-porous rate controlling membrane, allows the active inner reservoir layer to be coated and dried and provides that the active inner reservoir layer would not have to be laminated to a backing immediately following drying, as would be the practice. For example, the porous mesh film or non-porous rate-controlling membrane could be the temporary backing, or the porous mesh film could be embedded in the active reservoir coating. The features provided by the porous mesh film and the non-porous rate controlling membrane include:
structural integrity that would allow for continuous kiss-cutting and waste rewinding during manufacture, thereby increasing line speed and reducing defects;
differential release of the active film from a release liner, which allows for the use of two release liners of the same composition to perform the continuous process of the invention;
slowing the rate of seepage of the active ingredient or excipients from the active inner reservoir layer to the outer backing layers through the porous mesh film or the non-porous rate-controlling membrane, which would allow for at least temporary storage of the coated roll, allowing kiss-cutting and lamination with a backing roll, and
bonding of the active reservoir layer to the backing film.

Referring to the drawings, in which like numerals refer to like components or steps, there are disclosed broad aspects of various embodiments.

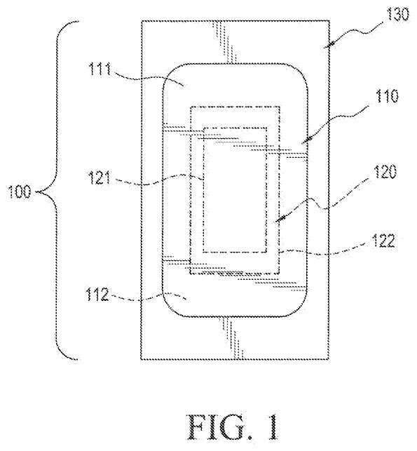

FIG. 1 shows the planar view of a double disk configuration of a dermal device for administration of active agents to the skin or mucosa of a host. The device 100 includes an outer disk patch 110, which includes an outer disk backing 111 and outer disk adhesive 112, an inner disk patch 120, which includes an inner disk active reservoir patch 121, and inner disk backing 122 which extends in all direction of the inner disk active reservoir 121, and a removable release liner 130. The outer disk patch 110, inner disk patch 120 and release liner 130 are planar layers and are assembled to one another to form a finished system composite of double disk configuration.

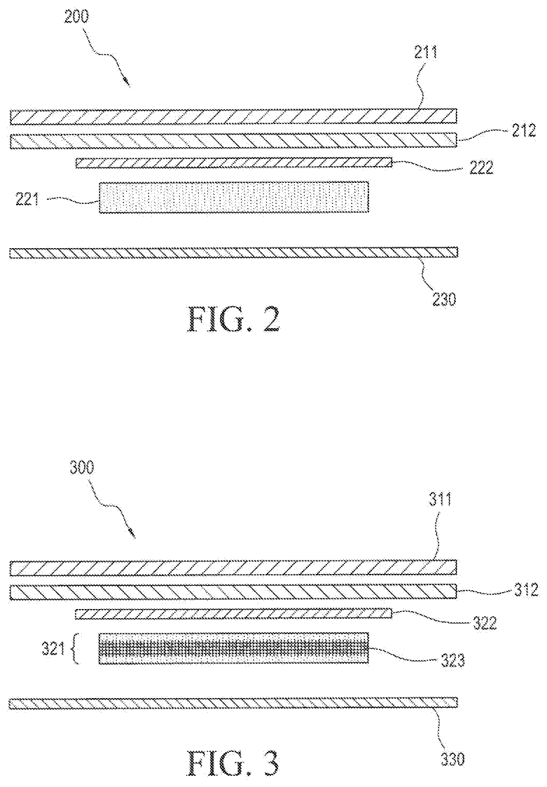

FIG. 2 shows a cross-sectional view of the dermal device 100 wherein the device contains an outer disk overlay backing 211 layered over an outer disk overlay adhesive 212, layered over an inner active reservoir layer backing 222 extending on all sides of an active reservoir layer 221 and further affixed to a removable release liner 230.

FIG. 3 illustrates an embodiment with an active reservoir layer 321 containing a porous mesh film 323 that brings structural integrity to the active reservoir layer.

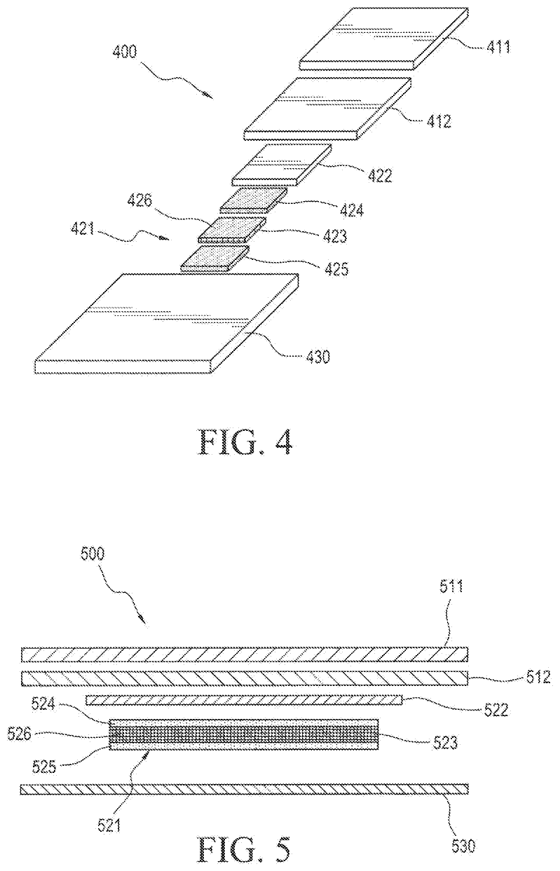

FIGS. 4 and 5 illustrate an active reservoir layer 421, 521 configured as two active adhesive layers 424, 524 and 425, 525 containing a porous mesh film 423, 523 or rate controlling membrane 426, 526 embedded therein.

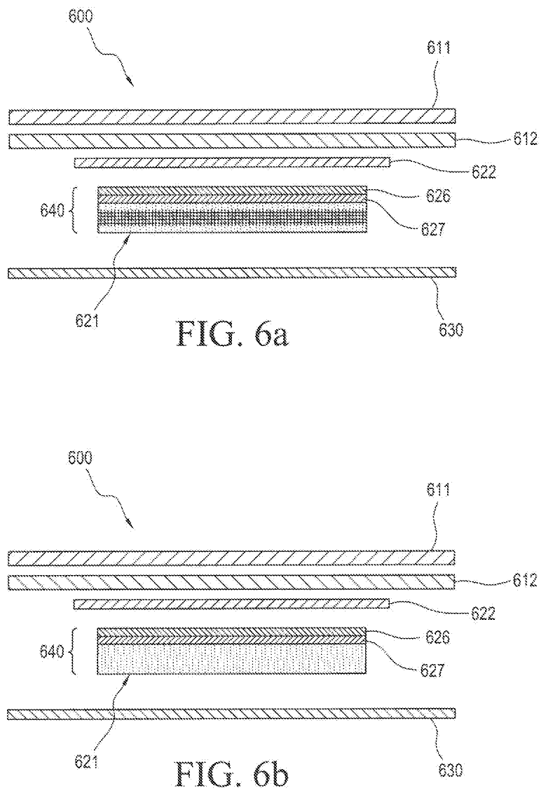

FIG. 6a illustrates an embodiment wherein the device contains an adhesive tie layer 626 and a backing film of the same size as the inner active reservoir layer 627 layered between the inner active reservoir layer backing 622 extending on all sides of the active reservoir layer 621 and the inner active reservoir layer 621. FIG. 6b illustrates an embodiment wherein a porous membrane is excluded.

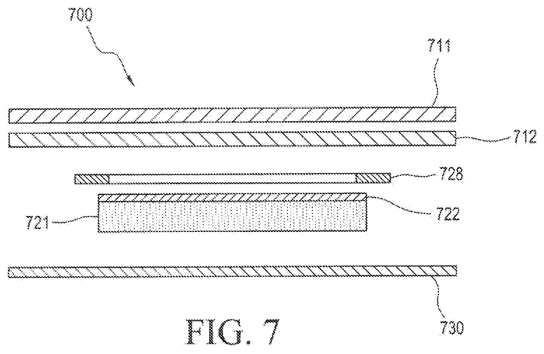

FIG. 7 illustrates an embodiment wherein the device contains a peripheral ring of backing film 728 layered in between the outer disk backing 711 and adhesive layer 712, and inner disk backing 722 which extends in all directions of the inner disk active reservoir 721.

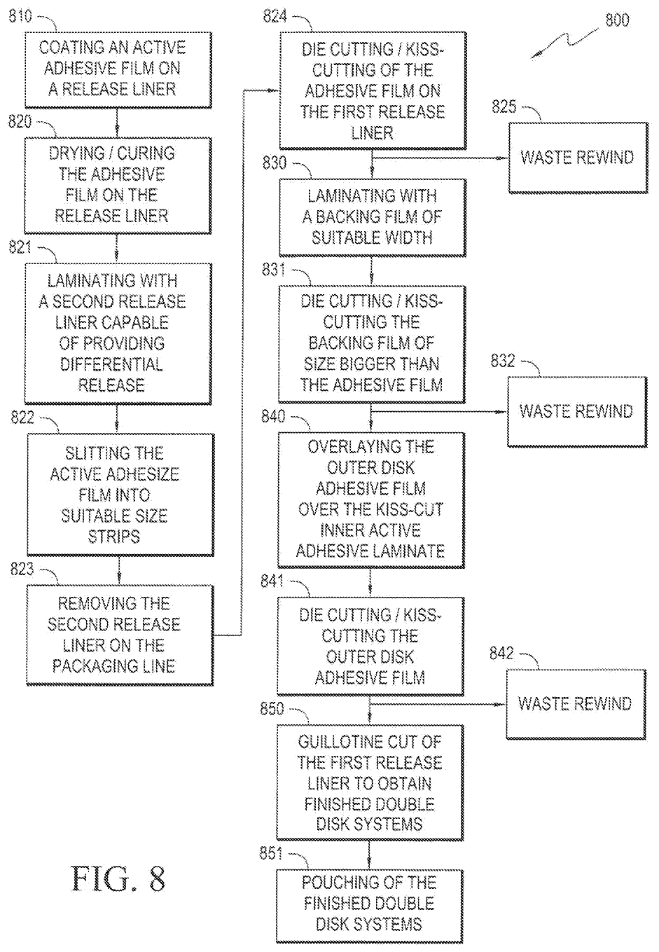

FIG. 8 illustrates an exemplary continuous process of manufacturing a dermal disk device. The method 800 begins in step 810 wherein the active adhesive film 121 is coated on a first release liner 130. In the next step 820, the adhesive-coated release liner is dried thereby fixing the active adhesive film 121 onto the first release liner 130. In the next step 821, a second release liner, capable of providing differential release is laminated onto the exposed side of the adhesive-coated release liner. In the next step 822, the dual release liner unit is slit into suitable size strips. In the next step 823, the second release liner is removed on the packaging line. In the next step 824, the remaining unit containing the active adhesive film 121 on the first release liner 130 is subjected to die-cutting and kiss-cutting. The resultant waste is rewound in step 825 without stoppage of the process using a structurally rigid membrane close to the dimension of resultant waste. In the next step 830 a backing film 122 of suitable width is laminated on the exposed side of the active adhesive film 121. In the next step 831, the backing film 122 is subjected to die-cutting and kiss-cutting to a size larger than the active adhesive film. The resultant waste is rewound in step 832 without stoppage of the process. In the next step 840, the outer disk adhesive film 110 is layered over the exposed side of the kiss-cut inner active adhesive laminate prepared in the previous step 831. In the next step 841 the outer disk adhesive film 110 is subjected to die-cutting and kiss-cutting. The resultant waste is rewound in step 842 without stoppage of the process. In the next step 850 the first release liner 130 is guillotine cut to obtain finished double disk systems 100. In the next step 851, the finished double disk systems 100 are packaged in suitable packaging.

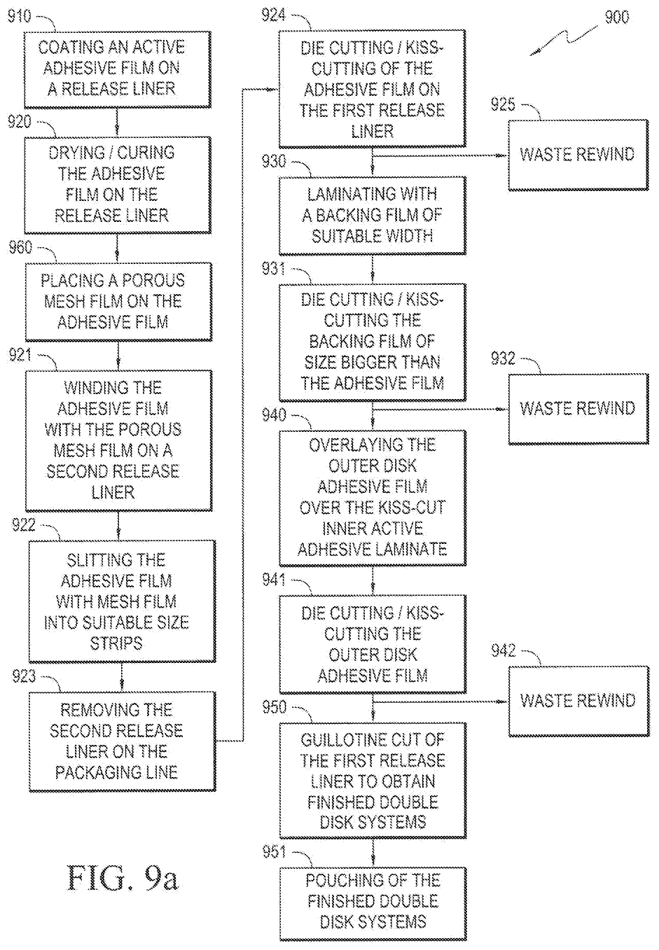

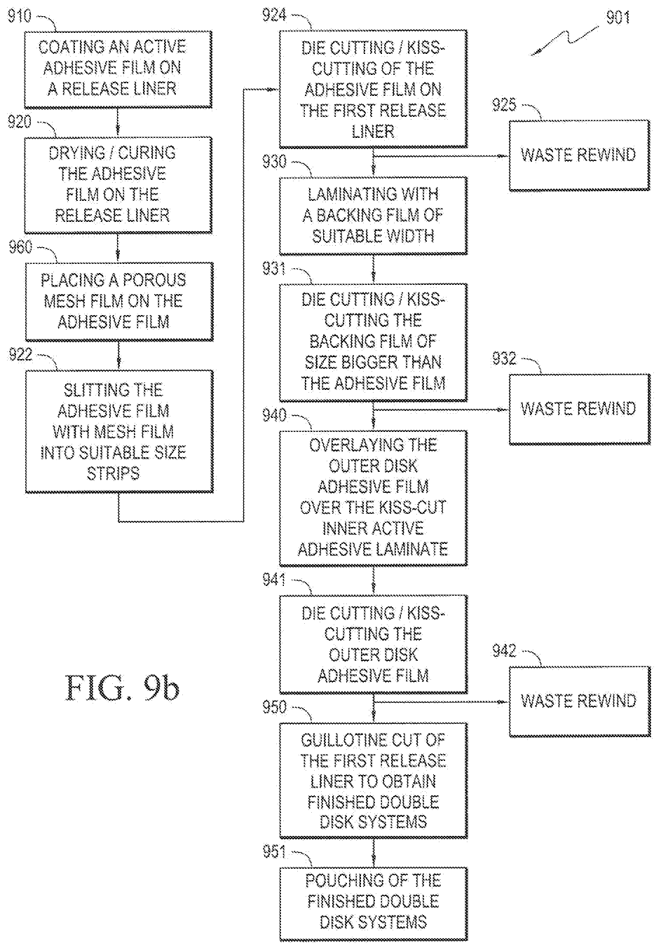

FIG. 9a illustrates an exemplary continuous process for manufacturing a dermal disk device. The method 900 begins in step 910 wherein the active adhesive film 121 is coated onto a first release liner 130. In the next step 920 the active adhesive-coated release liner is dried, thereby fixing the active adhesive film 121 on the release liner 130. In the next step 960, a porous mesh film 523 is placed on the active adhesive film 525 to form an active reservoir layer 521. In the next step 921, the exposed side of the active reservoir layer 521 is wound onto a second release liner. In the next step 922 the dual release liner unit is slit into suitable size strips. In the next step 923 the second release liner is removed on the packaging line. In the next step 924, the active reservoir layer 521 on the first release liner 530 is subjected to die-cutting and kiss-cutting. The resultant waste is rewound in step 925 without stoppage of the process. In the next step 930, a backing film 522 of suitable width is laminated onto the exposed side of the active reservoir layer 521. In the next step 931, the backing film 522 is subjected to die-cutting or kiss-cutting to a size larger than the active reservoir layer 521. The resultant waste is rewound in step 932 without stoppage of the process. In the next step 940 the outer disk adhesive film 512 is layered over the exposed side of the kiss-cut adhesive laminate prepared in previous step 931. In the next step 941, the outer disk adhesive film is subjected to die-cutting or kiss-cutting. The resultant waste is rewound in step 942 without stoppage of the process. In the next step 950, the first release liner 530 is guillotine cut to obtain finished double disk systems 500. In the next step 951, the finished double disk systems 500 are packaged.

FIG. 9b illustrates an exemplary continuous process for manufacturing a dermal disk device. The method 901 begins in step 910 wherein the active adhesive film 121 is coated onto a first release liner 130. In the next step 920 the active adhesive-coated release liner is dried, thereby fixing the active adhesive film 121 on the release liner 130. In the next step 960, a porous mesh film 523 is placed on the active adhesive film 525 to form an active reservoir layer 521. In the next step 922 the dual release liner unit is slit into suitable size strips. In the next step 924, the active reservoir layer 521 on the first release liner 530 is subjected to die-cutting and kiss-cutting. The resultant waste is rewound in step 925 without stoppage of the process. In the next step 930, a backing film 522 of suitable width is laminated onto the exposed side of the active reservoir layer 521. In the next step 931, the backing film 522 is subjected to die-cutting or kiss-cutting to a size larger than the active reservoir layer 521. The resultant waste is rewound in step 932 without stoppage of the process. In the next step 940 the outer disk adhesive film 512 is layered over the exposed side of the kiss-cut adhesive laminate prepared in previous step 931. In the next step 941, the outer disk adhesive film is subjected to die-cutting or kiss-cutting. The resultant waste is rewound in step 942 without stoppage of the process. In the next step 950, the first release liner 530 is guillotine cut to obtain finished double disk systems 500. In the next step 951, the finished double disk systems 500 are packaged.

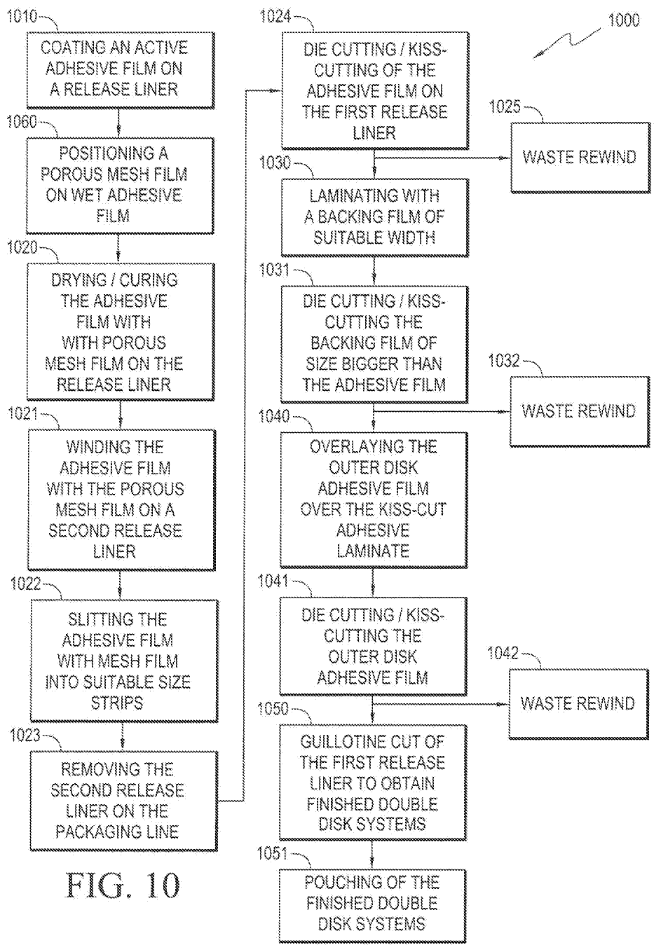

FIG. 10 illustrates an exemplary continuous process for manufacturing a dermal disk device, wherein the porous mesh film 523 is positioned on the active adhesive film before a drying step is performed. The method 1000 begins in step 1010 wherein the active adhesive film 525 is coated on a release liner 530. In the next step 1060, a porous mesh film 523 is positioned on the wet active adhesive film 525 to form an active reservoir layer. In the next step 1020, the active reservoir layer-coated release liner is dried or cured, thereby fixing the active reservoir layer 521 on the release liner. In the next step 1021, the exposed side of the active reservoir layer 521 is wound onto a second release liner. In the next step 1022, the dual release liner unit is slit into suitable size strips. In the next step 1023, the second release liner is removed on the packaging line. In the next step 1024, the active reservoir layer on the first release liner is subjected to die-cutting and kiss-cutting. The resultant waste is rewound in step 1025 without stoppage of the process. In the next step 1030, a backing film 522 of suitable width is laminated on the exposed side of the active reservoir layer 521. In the next step 1031, the backing film 522 is subjected to die-cutting and kiss-cutting to a size larger than the active reservoir layer 521. The resultant waste is rewound in step 1032 without stoppage of the process. In the next step 1040, the outer disk adhesive film 511 is layered over the kiss-cut adhesive laminate prepared in the previous step 1031. In the next step 1041, the outer disk adhesive film 511, is subjected to die-cutting and kiss-cutting. The resultant waste is rewound in step 1042 without stoppage of the process. In the next step 1050, the first release liner is guillotine cut to obtain finished double disk systems 500. In the next step 1051, the finished double disk systems 500 are packaged.

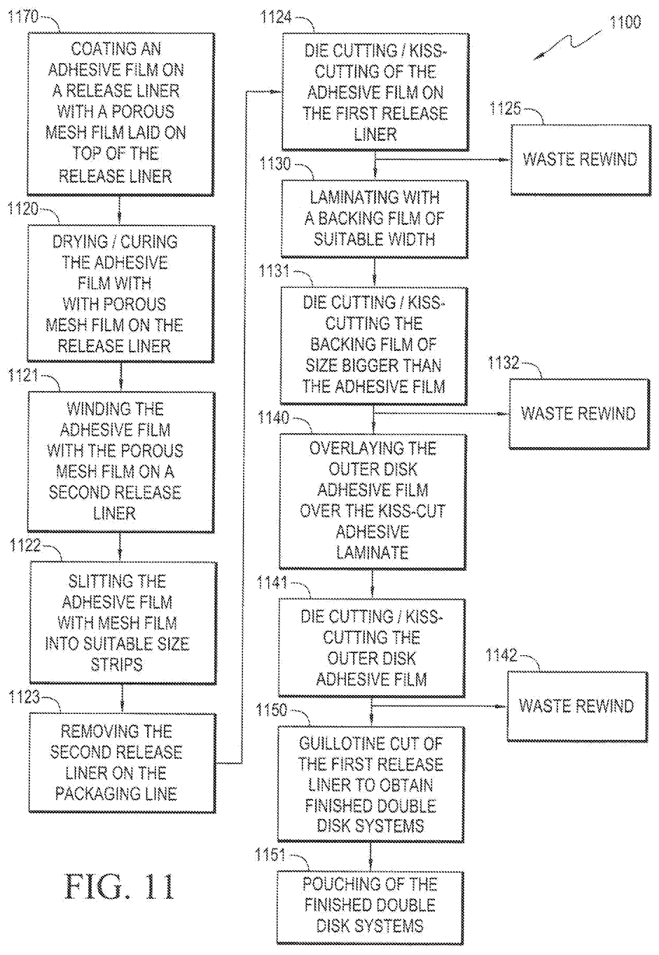

FIG. 11 illustrates an exemplary continuous process for manufacturing a dermal disk device. The method 1100 begins with step 1170, wherein an active adhesive film 524 is coated on a release liner 530 with a porous mesh film 523 laid on top of the release liner, wherein the active adhesive film 524 and porous mesh film 523 form an active reservoir layer 521. In the next step 1120, the active reservoir layer-coated release liner is dried or cured, thereby fixing the active reservoir layer 521 onto the release liner 530. In the next step 1121, the exposed side of the active reservoir layer 521 is wound onto a second release liner. In the next step 1122, the dual release liner unit is slit into suitable size strips. In the next step 1123, the second release liner is removed on the packaging line. In the next step 1124, the active reservoir layer 521 on the first release liner 530 is subjected to die-cutting and kiss-cutting. The resultant waste product is rewound in step 1125 without stoppage of the process. In the next step 1130, a backing film 522 of suitable width is laminated on the exposed side of the active reservoir layer 521. In the next step 1131, the backing film 522 is subjected to die-cutting and kiss-cutting to a size larger than the active reservoir layer 521. The resultant waste is rewound in step 1132 without stoppage of the process. In the next step 1140, the outer disk adhesive film 511 is layered over the kiss-cut backing laminate film 522. In the next step 1141, the outer disk adhesive film 511 is subjected to die-cutting and kiss-cutting. The resultant waste is rewound in step 1142 without stoppage of the process. In the next step 1150, the first release liner 530 is guillotine cut to obtain finished double disk systems 500. In the next step 1151, the finished double disk systems 500 are packaged.

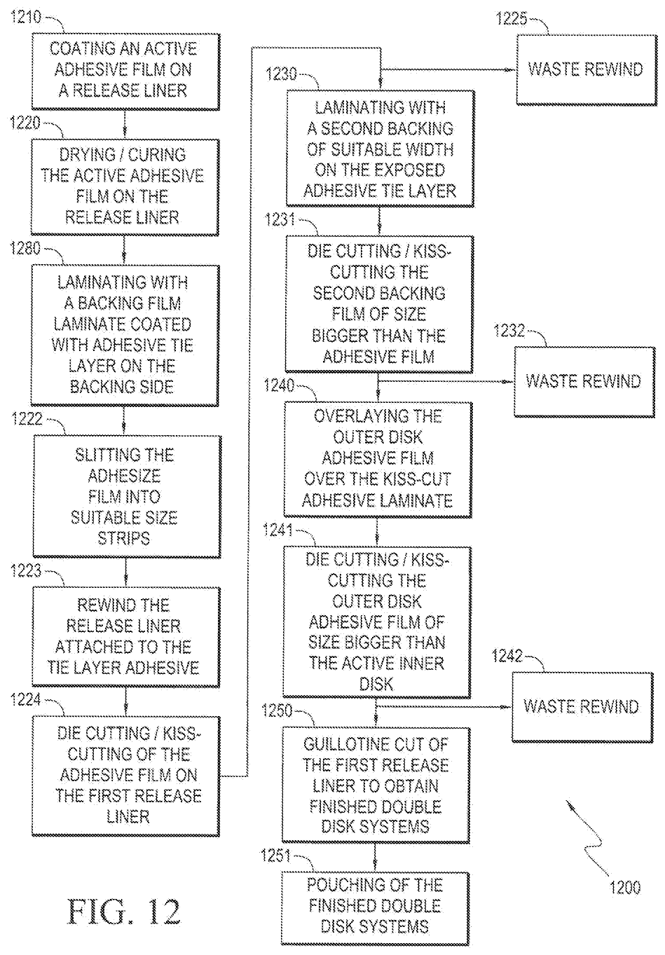

FIG. 12 illustrates an exemplary continuous process for manufacturing a dermal disk device. The method 1200 begins with the step 1210 of coating an active adhesive film 621 on a release liner 630. In the next step 1220, the active adhesive film 621 is dried or cured on the release liner 630. In the next step 1280, a backing film laminate 627 coated with an adhesive tie layer 626 on the backing side is laminated onto the exposed side of the active adhesive film 621. The backing film laminate contains a backing film, adhesive tie layer and a second release liner layered on top of the adhesive tie layer. In the next step 1222, the dual release liner unit is slit into suitable size strips. In the next step 1223, the second release liner attached to the tie layer adhesive is rewound to expose the inner disk reservoir 640. In the next step 1224, the inner disk reservoir on the first release liner formed in the previous step 1223, is subjected to die-cutting and kiss-cutting. The resultant waste is rewound in step 1225 without stoppage of the process. In the next step 1230, a second backing film 611 of suitable width is laminated on the exposed adhesive tie layer 626. In the next step 1231, the second backing film 622 is subjected to die-cutting and kiss-cutting to a size larger than the active inner disk reservoir 640. The resultant waste is rewound in step 1232 without stoppage of the process. In the next step 1240, the outer disk adhesive film 611 is layered over the kiss-cut active inner disk patch prepared in the previous step 1231. In the next step 1241, the outer disk adhesive film 622 is subjected to die-cutting and kiss-cutting to a size larger than the active inner disk patch formed in step 1231. The resultant waste is rewound in step 1242 without stoppage of the process. In the next step 1250, the first release liner 630 is guillotine cut to obtain finished double disk systems 600. In the next step 1251, the finished double disk systems 600 are packaged.

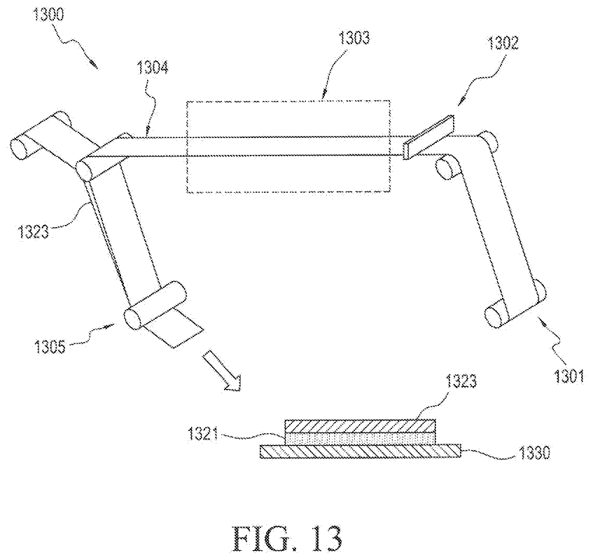

In the diagrammatic illustration of FIG. 13, the process 1300 begins with step 1301 wherein the release liner is wound upward into the process. In the next step 1302, the active adhesive film solution is coated at the coating head onto the release liner. In the next step 1303, the film is dried in a drying oven to produce a dried film 1304. The porous membrane material 1323 is wound over the dried film in step 1305 to produce a unit having a porous membrane 1323 layered over an active adhesive film 1321 layered over a release liner 1330.

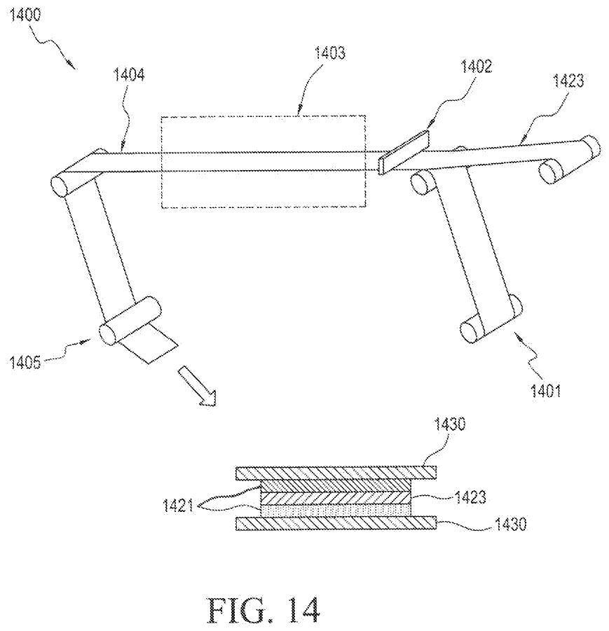

In the diagrammatic illustration of FIG. 14, the process 1400 begins with step 1401 wherein the release liner 1430 is wound upward into the process. In the next step 1402, an active adhesive film solution 1421 is coated onto the release liner along with a porous membrane film 1423 that is simultaneously wound into the process. The active and porous membrane coated release liner is dried in a drying oven 1403 to form a dried film with porous membrane 1404, wherein the porous membrane 1423 is embedded within active adhesive layer 1421 and wherein active adhesive layer 1421 is layered onto a second release liner 1430.

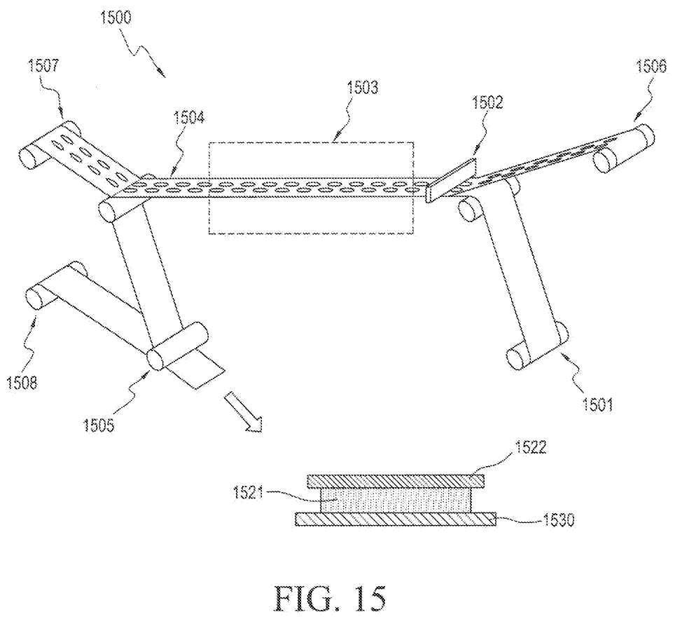

In the diagrammatic illustration of FIG. 15, the process 1500 begins with step 1501 wherein the release liner is wound up into the process. In the next step 1502, an active adhesive film solution is coated onto the release liner along with a template with a die-cut inner disk area 1506, which is simultaneously wound into the process. The active and template-coated release liner is dried in a drying oven 1503 to produce a dried film 1504. A waste rewind step 1507 is performed wherein the template is removed from the process. The dried film is wound downward into contact with an inner disk backing roll to form a unit having an inner disk backing 1522 layered over an inner active matrix 1521 layered onto a release liner 1530.

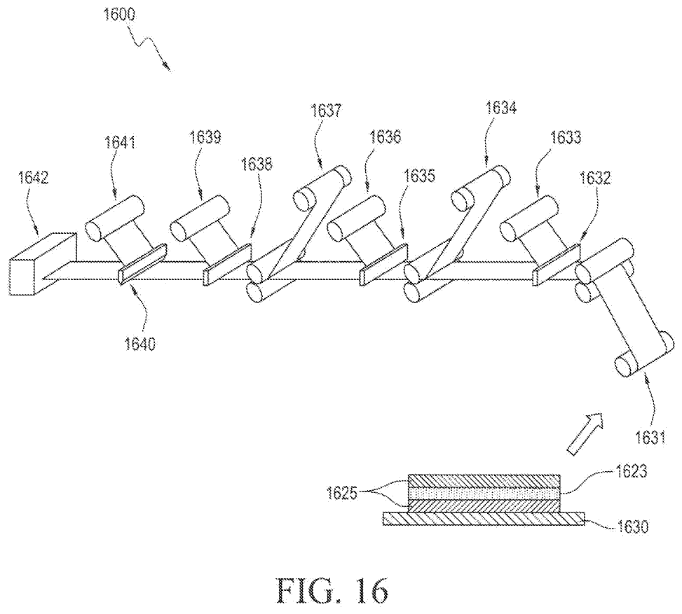

In the diagrammatic illustration of FIG. 16, the active inner reservoir laminate 1421, 1423 prepared by the process illustrated in FIG. 14, is further wound up into the process in step 1631 and is kiss-cut in step 1632. A first waste rewind is performed in step 1633 and the inner active layer backing 1634 is laminated onto the exposed side of the active inner reservoir layer. The inner active backing layer is kiss-cut in step 1635 and a second waste rewind 1636 is performed without stoppage of the process. In the next step 1637, the outer disk laminate is laminated onto the exposed surface of the inner active layer backing and the inner active layer backing is further kiss-cut in step 1638. A third waste rewind step 1639 is performed without stoppage of the process and the release liner is then die cut in step 1640. A fourth and final waste rewind step 1641 is performed without stoppage of the process and the finished dermal device 100 is packaged in step 1642.

In various embodiments, the inner disk reservoir layer 221 and/or the outer disk layer 110 may be prepared using a hot melt process or a solvent based process such as solvent coating, curing or cross-linking.

In various embodiments, suitable porous mesh films include degradable or non-degradable polymeric materials, polyester, polypropylene, polyethylene, nylon, cellulose, acrylate, glass fiber, PET, PES, PVDF, PC, PTFE, MCE or mixture of material meshes. Suitable polymeric materials include synthetic polymers and bio-polymers. The porous mesh could be manufactured into a film structure which could be of different thickness or porosity depending on the functionality to be obtained from the mesh in addition to bringing structural integrity. The porous mesh could be multilayer or monolayer mesh film. The porous mesh could be polymeric or non-polymeric mesh film or could be woven or non-woven. Typical examples of useful meshes include Reemay® spunbond polyester, Typar® spunbond polypropylene, nylon mesh. Advantageous material that function as porous mesh satisfy the minimum requirement in that the tensile strength of the dried inner disk reservoir layer with mesh is greater than the tensile strength of dried inner disk reservoir layer without the mesh. The thickness of the porous mesh is based on providing sufficient integrity for processability. The tensile strength of the porous mesh is based on providing sufficient integrity for processing in the machine direction.

In various embodiments, the active inner reservoir layer 121 can contain additional excipients that include enhancers that promote the penetration of the active agent through the skin. The enhancer may be incorporated into the active inner reservoir layer 121 by solvent blending or melt-blending. Suitable enhancers include monovalent, saturated and unsaturated aliphatic and cycloaliphatic alcohols having 6 to 12 carbon atoms such as cyclohexanol, lauryl alcohol and the like; aliphatic and cycloaliphatic hydrocarbons such as mineral oils; cycloaliphatic and aromatic aldehydes and ketones such as cyclohexanone; N,N-di (lower alkyl) acetamides such as N,N-diethyl acetamide, N,N-dimethyl acetamide, N-(2-hydroxyethyl) acetamide, and the like; aliphatic and cycloaliphatic esters such as isopropyl myristate and lauricidin; N,N-di (lower alkyl) sulfoxides such as decylmethyl sulfoxide; essential oils; nitrated aliphatic and cycloaliphatic hydrocarbons such as N-methyl-2-Pyrrolidone, Azone; salicylates, polyalkylene glycol silicates; aliphatic, keto or aromatic acids such as oleic acid and lauric acid, terpenes such as cineole, surfactants such as sodium lauryl sulfate, siloxanes such as hexamethyl siloxane; mixtures of the above materials; and the like.

In various embodiments, suitable non-porous rate-controlling membranes include degradable or non-degradable polymeric materials such as spunbound polyester fabric. Suitable polymeric materials include synthetic polymers and bio-polymers. Other examples of degradable polymers are poly-caprolactone, polysaccharide based polymers, celluloses, biopolyesters, polylactids, polyesteramides, aliphatic or aromatic copolyesters, gums, chitosan, starches and the like.

In various embodiments, the backing layer is composed of a material or combination of materials that is substantially impermeable to the layer or layers with which it can be in contain, e.g., the active reservoir layer 121 and the active agents or ingredients contained therein. By impermeable, it is meant that the other components in contact with the backing layer or component under consideration will not appreciably permeate through such layer or component for the normal period of use and storage of the device. Some suitable materials for the backing layer include, for example, cellophane, cellulose acetate, ethyl cellulose, plasticized vinyl acetate-vinyl chloride copolymers, ethylene-vinyl acetate copolymer, polyethylene terephthalate, polyvinyl chloride, nylon, polyethylene, polypropylene and polyvinylidene chloride (e.g., SARAN) and polyolefin.

In various embodiments, the release liner is composed of materials suitable for use in the backing layer provided they are active agent impermeable. Such materials as a release liner are made removable or releasable from the adhesive layers or active agent layers by, for example, conventional treatment with silicon, Teflon or other suitable coating on the surface thereof. In one embodiment, the first and second release liners utilized in the continuous process for preparing the dermal device of the invention are composed of the same release coating. In another embodiment, the first and second release liners utilized in the continuous process for preparing the dermal device of the invention are composed of different release coatings.

In various embodiments, suitable inner disk active agents include; high melting and low melting point pharmacological or non-pharmacological agents. Active agents are preferably selected from the group consisting of pharmaceutical agents that provide pharmacological action to a host and are not limited to chemical or pharmacological functionality.

In various embodiments, abuse deterrent agents may be included in the active reservoir layer 121, adhesive tie layer 626 or outer disk layer 110. Suitable abuse deterrent substances include opioid antagonists, absorbent materials, such as activated charcoal, magnesium aluminum silicate, or activated alumina; bittering agents, such as sucrose octaacetate, denatonium saccharide, denatonium benzoate, caffeine, quinine (or a quinine salt such as quinine sulfate), bitter orange peel oil, and other botanical extract ingredients, such as pepper extract (Cubeb), capsicum, and the like and indicator dyes, such as allura red, amaranth, brilliant blue, canthaxanthin, carmine, carmoisine, carotene, curcumin, erythrosine, green S, indigo carmine, iron oxide black, iron oxide red, iron oxide yellow, patent blue, phloxine O, ponceau 4R, quinoline yellow, riboflavin, sunset yellow, tartrazine, titanium dioxide, vegetable carbon black, and other natural colors such as annatto, beet, black carrot, black currant, caramel, carmine, carmine lake, chlorophyll, cochineal, elderberry, grapeskin/grape juice, malt, paprika, red cabbage, turmeric, and anthocyanins and tonicity-increasing agents such as carbohydrates (e.g., dextrose, lactose, etc.), salts, mannitol, urea, acids (e.g., tartaric acid), and combinations thereof. The substances are to be incorporated into the dosage forms of the abuse-potential drugs in such a manner that the deterrent substance does not exhibit its deterrent effect when a dosage form of the drug is properly administered, but exhibits a deterrent effect when the dosage form is chewed, crushed or chemically extracted for nasal (snorting), inhalation (smoking), oral, buccal or sublingual administration or injected.

The inner disk active reservoir layer may include a reservoir of pressure sensitive adhesive or non-adhesive polymeric matrices which provide active agent release when administrated to a host. Suitable pressure sensitive adhesives provide physical and chemical stability and are selected based on the properties of the active agent. For example, silicone adhesives with reduced hydroxy groups would be preferred for amine group-containing active agents.

In various embodiments, suitable pressure-sensitive adhesive materials include some natural rubber and synthetic rubber adhesives and cross-linkable laminating adhesives. Examples of suitable natural rubber adhesives include R-1072 from B.F. Goodrich Co., No. 735 from C. L. Hathaway, and No. 5702 from Evans St. Clair. Examples of synthetic rubber adhesives include Jowatherem 270-00 and Jowatherem S-3202 from Jowat Corp. and 70-9416 from National Starch. Other suitable laminating adhesives include the Dow Corning laminating silicone adhesives and the Lord Corporation Tycel 7900 series laminating adhesives. Also contemplated are acrylic copolymers such as those available from National Starch and Chemical Co. of Bridgewater, N.J. under the marks DURO-TAK 87-2516 and DURO-TAK 87-2287. The adhesives most impermeable to most active ingredients are cross-linkable laminating adhesives, which are well-known to those of ordinary skill in the art.

The active agent adhesive layers may be pressure-sensitive adhesives. Any of the well-known, dermatologically acceptable, pressure-sensitive adhesives which permit drug migration therethrough can be used in the present invention. Some suitable permeable adhesives include acrylic or methacrylic resins such as polymers of alcohol esters of acrylic or methacrylic acids and alcohols such as n-butanol, isopentanol, 2-methylbutanol, 1-methyl-butanol, 1-methyl-pentanol, 2-methylpentanol, 3-methylpentanol, 2-ethyl-butanol, isooctanol, n-decanol, or n-dodecanol, alone or copolymerized with ethylenically unsaturated monomers such as acrylic acid, methacrylic acid, acrylamide, methacrylamides, N-alkoxymethyl acrylamides, N-alkoxymethyl methacrylamides, N-t-butyl-acrylamide, itaconic acid, vinyl acetate, N-branched alkyl maleamic acids wherein the alkyl group has 10-24 carbon atoms, glycol diacrylates, or mixtures of these monomers; polyurethane elastomers; vinyl polymers such as polyvinyl alcohol, polyvinyl ethers, polyvinyl pyrrolidone, and polyvinyl acetate; urea formaldehyde resins; phenol formaldehyde resins, resorcinol formaldehyde resins; cellulose derivatives such as ethylcellulose, methylcellulose, nitrocellulose, cellulose acetate butyrate and carboxymethylcellulose; and natural gums such as guar, acacia, pectina, starch, destria, gelatin, casein, etc.

Other suitable pressure-sensitive adhesives include polyisobutylene pressure sensitive adhesives, rubber pressure-sensitive adhesives, cross-linked adhesives, silicone pressure-sensitive adhesives or combination of adhesives. The adhesives may also be compounded with tackifiers and stabilizers as is well-known in the art. Adhesives that are preferred for their active agent permeability include acrylic copolymer adhesives such as Avery Chemical Company's AS-351 HSX, preferably at a coating weight of between 75 and 125 g/m². This pressure-sensitive adhesive is a cross-linkable polymer which provides a permanently tacky film having a total solids content of about 52%, Brookfield viscosity (LVT/Spindle No. 4/12 RPM @ 25′ C) of from about 15,000 to 25,000 cps. at a weight per gallon of about 7.4 lbs. It can also be diluted with hexane or toluene to a desired solids and/or viscosity range, particularly for use in conventional coating equipment.

Other such adhesives that can also be used for these purposes include an acrylic pressure-sensitive adhesive sold by National Starch and Chemical Co. under the designation DURO-TAK 80-1054. This adhesive has a solids content of 47.5%, a viscosity of 3,000 cps., and plasticity (Williams) of 2.9 mm. It is generally used with a solvent system including ethyl acetate, heptane, isopropyl alcohol and toluene. Another such adhesive is sold by the UCB Group under the designation GELVA Multipolymer Emulsion 2484, and comprises a stable aqueous acrylic emulsion pressure-sensitive adhesive having a solids content of 59% and a viscosity of 1,500 to 2,300 cps. Examples of other acrylic adhesives include Gelva 788 and 733 from UCB, PS-41 from C.L.-Hathaway, Vr-0833 from H.B. Fuller, Adcot 73A207A from Morton Chemical, Nos. 80-2404, 80-1054, 72-9056 and 72-9399 from National Starch, Nos. E-2015, E-2067 and E-1960 from Rohm & Haas, M-6112 from Uniroyal, Inc. and Daratak 74 L from W.R. Grace. Suitable rubber adhesives include Duro-Tak 36-6172 from National Starch and Morstik 118 from Morton Chemical. An example of a suitable silicone adhesive is 7-4502 from Dow Corning. Adhesive polymers are preferably selected based on suitable product design to minimize residual drug and are not limited to adhesive chemical functionality.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

In various embodiments, suitable outer disk layers consist of a backing, at least one adhesive layer and removable release liner. Suitable materials for outer disk layer backing include woven or non-woven, fabric, spun-laced, spun-bonded, multi-layered, porous or non-porous materials. Suitable adhesive include solvent coated-cross-linked, un-crosslinked, cross-linkable or thermos-plastic adhesives. The adhesives are preferably selected from groups that provide comfortable and adequate wear characteristics and not limited to chemical functionality of the adhesive or mixtures of adhesives.

The removable release liner suitable for the system include but is not limited to silicone coated, fluoro-carbon coated, uv cross-linked, epoxy coated liners suitable for providing low peel force selected based on the adhesive choice of the inner and outer disk layer and not limited to chemical functionality or the substrate.

### Example 1

In the following example, a study was conducted to compare various double disk transdermal systems in regards to the migration of excipients into the outer adhesive overlay from the inner reservoir layer. Transdermal systems Sample 2 and Sample 4 are representative embodiments of the dermal devices of the invention.

In this study, a placebo inner reservoir film was used containing enhancer and a plasticizer excipients The inner reservoir film was prepared by weighing 13.205 gm of ethyl acetate, 28.5 gm of ethanol, 6.5 gm of enhancer, 13 gm of plasticizer, 10 gm of Povidone and 128.7 gm of Duro-Tak acrylate adhesive solids. The blend was mixed overnight and coated on a suitable release liner and dried in ovens to obtain a nominal coat weight of 80 g/m². Portion of the dried laminate were either laminated with a PET backing or were laminated with a second release liner to produce the gap design devices (Sample 2 and Sample 4). The portion of the laminate with was laminated on PET backing was die cut and the backing side was placed on the outer adhesive overlay. The outer adhesive overlay was die cut and the inner disk release liner was removed to obtain the double disk design where the inner disk reservoir would be in contact with the outer disk adhesive overlay (Sample 3 and sample 5). For the portion of the laminate which was made to produce the oversized backing Gap design devices of the invention, the inner disk reservoir was die cut and placed on a PET backing film after the release liner was removed. The PET backing film was die cut to a size larger than the inner disk reservoir film. The backing side of the die cut film was adhered to the outer disk adhesive overlay. This resulted in the inner disk not being in contact with the outer disk adhesive overlay in all directions. The finished systems were stored for 1 month at 60° C. in an oven and the outer disk was separated from the inner disk reservoir to determine the extent of migration of enhancer and plasticizer.

Specific designs tested for migration of excipients in the outer adhesive overlay from the inner reservoir layer:

Excipient migration from inner disk to outer disk study (oversized backing and double disk design comparison)

Sample 1—Placebo inner disk only (PET backing, 80 g/m², removable release liner)
Sample 2—oversized backing design (Placebo inner disk on larger area PET backing, with, outer disk acrylate adhesive 1 layer)
Sample 3—double disk design—(Placebo inner disk on same sized PET backing, on, outer disk acrylate adhesive 1 layer)
Sample 4—oversized backing design (Placebo inner disk only on larger area PET backing, on outer disk acrylate adhesive 2 layer)
Sample 5—double disk design (Placebo inner disk on same sized PET backing, on outer disk acrylate adhesive 2 layer).

Enhancer Assay

| | Initial | 1 month |
|---|---|---|
| Sample 2 (Oversized) | | |
| Inner Disk | 112.1% | 87.9% |
| Outer Disk | | 5.3% |
| Total | | 93.2% |
| Sample 3 | | |
| Inner Disk | 112.1% | 75.2% |
| Outer Disk | | 8.1% |
| Total | | 83.3% |
| Sample 4 (Oversized) | | |
| Inner Disk | 112.1% | 83.3% |
| Outer Disk | | 5.7% |
| Total | | 89.0% |
| Sample 5 | | |
| Inner Disk | 112.1% | 73.8% |
| Outer Disk | | 8.0% |
| Total | | 81.8% |

Plasticizer Assay

| | Initial | 1 month |
|---|---|---|
| Sample 2 (Oversized) | | |
| Inner Disk | 118.9% | 111.6% |
| Outer Disk | | 1.6% |
| Total | | 113.2% |
| Sample 3 | | |
| Inner Disk | 118.9% | 101.8% |
| Outer Disk | | 5.7% |
| Total | | 107.5% |
| Sample 4 (Oversized) | | |
| Inner Disk | 118.9% | 112.6% |
| Outer Disk | | 1.9% |
| Total | | 114.5% |
| Sample 5 | | |
| Inner Disk | 118.9% | 91.9% |
| Outer Disk | | 9.0% |
| Total | | 100.9% |

The results demonstrate that the oversized backing devices of the invention had lower migration of excipients compared to the conventional double disk design. The migration of enhancer in the outer adhesive overlay at 1 month, 60° C. was 5.3% for the oversized backing design compared to 8.1% for conventional double disk design. The migration of plasticizer in the outer adhesive overlay at 1 month, 60° C. was 1.6% for the oversized backing design compared to 5.7% for the conventional double disk design systems.

What is claimed is:

1. A dermal device for administration of one or more active agents to the skin or mucosa, the device comprising:
a disk, comprising a reservoir layer comprising
a film layer having a first surface and a second surface, wherein the first surface is opposite the second surface and the film layer is comprised of a material that brings structural integrity to the reservoir layer; and
a first adhesive layer on the first surface of the film layer and a second adhesive layer on the second surface of the film layer, wherein the film layer is embedded between the first adhesive layer and the second adhesive layer;
a backing overlay adjacent to the disk extending beyond the perimeter of the disk; and
a removable release liner.

2. A process for preparing a dermal device, the process comprising the steps of:
fixing a first layer to a first release liner;
placing a second material adjacent to the first layer to form an active inner reservoir layer, wherein the second material brings structural integrity to the active inner reservoir layer;
winding the exposed side of the active inner reservoir layer on a second release liner to form a dual release liner unit;
kiss-cutting the active inner reservoir layer into suitable size patches; and
laminating a backing layer extending beyond the perimeter of the active inner reservoir layer on the exposed side of the active inner reservoir layer,
wherein the process is performed using a continuous manufacturing process.

3. The process of claim 2, wherein the first release liner and the second release liner are comprised of the same release coating chemistry.

4. The process of claim 2, wherein the first release liner and the second release liner are comprised of different release coating chemistry.

5. The process of claim 2, wherein the second material is a porous mesh film.

6. The process of claim 2, wherein the porous mesh film comprises a material selected from the group consisting of polyester, polypropylene, polyethylene, nylon, cellulose, acrylate, glass fiber, polyethylene terephthalate, polyethersulfone, polyvinylidene fluoride, polycarbonate, polytetrafluoroethylene, mixed cellulose ester, and mixtures thereof.

7. The process of claim 2, wherein the second material is a nonporous film.

8. The process of claim 7, wherein the nonporous film comprises a material selected from the group consisting of degradable and non-degradable polymer materials.

9. The process of claim 8, wherein the polymer material is selected from the group consisting of spunbound polyester fabrics, polyethylene, poly-caprolactone, polysaccharide based polymers, celluloses, biopolyesters, polylactides, polyesteramides, aliphatic or aromatic copolyesters, gums, chitosan, starches and mixtures thereof.

10. The process of claim 2, further comprising fixing a second layer to the second material, wherein the second material is located between the first layer and the second layer.

11. The process of claim 2, wherein the step of fixing a first layer to a first release liner comprises coating a wet adhesive film on the first release liner.

12. A process for preparing a dermal device, the process comprising the steps of:
coating a wet adhesive film on a first release liner;
drying the wet adhesive film on the first release liner to form an active inner reservoir layer;
laminating the active inner reservoir layer with a first backing film layer coated with an adhesive tie layer that brings structural integrity to the waste rewind;
winding the exposed side of the adhesive tie layer on a second release liner to form a dual release liner unit;
removing the second release liner to expose one side of the adhesive tie layer;
kiss-cutting the laminated active inner reservoir layer into suitable size patches;
laminating a second backing layer extending beyond the perimeter of the active inner reservoir layer on the exposed side of the adhesive tie layer; and
laminating the second backing layer with an outer disk adhesive layer,
wherein the process is performed using a continuous manufacturing process.

13. The process of claim 12, wherein the first release liner and the second release liner are comprised of the same release coating chemistry.

14. The process of claim 12, wherein the first release liner and the second release liner are comprised of different release coating chemistry.
